(19) United States
(12) Patent Application Publication
(10) Pub. No.: US 2013/0030267 A1
(43) Pub. Date: Jan. 31, 2013

(54) MULTI-PURPOSE SENSOR SYSTEM

(71) Applicants: Daniel Lisogurski, Boulder, CO (US); Andy S. Lin, Boulder, CO (US); Friso Schlottau, Lyons, CO (US)

(72) Inventors: Daniel Lisogurski, Boulder, CO (US); Andy S. Lin, Boulder, CO (US); Friso Schlottau, Lyons, CO (US)

(73) Assignee: Nellcor Puritan Bennett LLC, Boulder, CO (US)

(21) Appl. No.: 13/193,870

(22) Filed: Jul. 29, 2011

(51) Int. Cl. A61B 5/1468

(52) U.S. Cl. 600/324

(57) ABSTRACT

Embodiments of the present disclosure relate to multi-purpose sensors for monitoring a plurality of physiological parameters. According to certain embodiments, the multi-purpose sensors may include optical elements for determining oxygen saturation and regional saturation. In additional embodiments, such sensor may include multiple electrodes that are configured for bispectral index monitoring. In particular embodiments, portions of the multi-purpose sensors may be removed and discarded when no longer needed.

### BACKGROUND

The present disclosure relates generally to medical devices and, more particularly, to multi-purpose sensors for determining physiological parameters, such as plethysmographically-determined parameters and electroencephalography-derived parameters,

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring certain physiological characteristics of a patient Such devices provide doctors and other healthcare personnel with the information they need to provide the best possible healthcare for their patients. As a result, such monitoring devices have become an indispensable part of modern medicine. For example, photoplethysmography is a common technique for monitoring physiological characteristics of a patient, and one device based upon photoplethysmography techniques is commonly referred to as a pulse oximeter. Pulse oximeters may be used to measure and monitor various blood flow characteristics of a patient. A pulse oximeter may be utilized to monitor the blood oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

A patient in a hospital setting may be monitored by a variety of medical devices, including devices based on pulse oximetry techniques. For example, a patient may be monitored with a pulse oximetry device, which may appropriate for a wide variety of patients. Depending on the patient's clinical condition, a physician may also monitor a patient with a regional saturation monitor placed on the patient's head to determine if the patient is at risk of hypoxia. If a patient is scheduled for surgery, additional monitoring devices may be applied. One such device may include a sensor for bispectral index (BIS) monitoring to measure the level of consciousness by algorithmic analysis of a patient's electroencephalography (EEG) during general anesthesia. Examples of parameters assessed during the BIS monitoring may include the effects of anesthetics, evaluating asymmetric activity between the left and right hemispheres of the brain in order to detect cerebral ischemia, and detecting burst suppression. Such monitoring may be used to determine if the patient's anesthesia level is appropriate and to maintain a desired anesthesia depth.

Proper medical sensor placement may be complex if multiple sensors (e.g., pulse oximetry and regional saturation sensors) are used on the patient's tissue. Each type of sensor may include its own cable and, in some instances, its own dedicated monitor. Accordingly, the sensors or their monitors may physically interfere with one another. Further, depending on the arrangement of the sensors with respect to one another, the sensors may also electrically or optically interfere with one another, causing signal artifacts. In addition, certain types of sensors are relatively large and are configured for a particular geometric configuration on the tissue. For example, during BIS monitoring, multiple electrode sensors are applied directly to a patient's skin to acquire the EEG signal. Because BIS monitoring sensors are applied for patient monitoring during specific medical procedures, preexisting medical sensors already in place (e.g., oximetry sensors) may occupy a preferred BIS sensor location on the patient's tissue. While the sensors may be repositioned to accommodate the BIS sensor, such repositioning may affect the adhesion of the sensor to the skin. Further, the active elements of each sensor may be surrounded by a light-blocking material such as adhesive foam, and it may be difficult to position multiple sensors in their preferred locations without removing or cutting back the light-blocking material around the active elements.

### BRIEF DESCRIPTION OF THE DRAWINGS

Advantages of the disclosed techniques may become apparent upon reading the following detailed description and upon reference to the drawings in which:

FIG. 1 is a front view of an embodiment of a monitoring system configured to be used with a multi-purpose sensor for oximetry and regional saturation;

FIG. 2 is a block diagram of the monitoring system of FIG. 1;

FIG. 3 is a top view of an embodiment of a multi-purpose sensor configured to be used in conjunction with the monitoring system of FIG. 1;

FIG. 4 is a top view of an alternative embodiment of a multi-purpose sensor configured to be used in conjunction with the monitoring system of FIG. 1;

FIG. 5 is a view of a monitoring system including a multi-purpose sensor that includes BIS sensor functionality;

FIG. 6 is a perspective view of a multi-purpose sensor configured to be used in conjunction with the monitoring system of FIG. 5 applied to a patient;

FIG. 7 is a top view of a multi-purpose sensor with a removable portion;

FIG. 8 is a top view of the multi-purpose sensor with a BIS monitoring portion removed from an optical portion;

FIG. 9 is a top view of an alternative multi-purpose sensor including three separate portions; and

FIG. 10 is an example of an indicator for providing an electrical feedback related to sensor configuration.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure is generally directed to multi-purpose combination sensors and techniques for electroencephalography (EEG) and/or photoplethysmography. Such multi-purpose sensors may include optical elements capable of performing pulse oximetry and regional saturation measurements. Such multi-purpose sensors may also include EEG electrodes for BIS monitoring and optical elements for photoplethysmography. Further, the sensors may include features to facilitate proper placement of the sensor on the tissue and proper placement of the sensing features (i.e., the electrodes and optical elements) with respect to one another. As discussed herein, the multi-purpose sensor configurations may also include features that reduce interference between the electrodes and the optical elements. For example, such features may include relative positioning on the sensor that reduces cross-talk and/or optical interference. In other embodiments, the features may be embodied on a monitoring system configured for use with the combination sensors. For example, a monitor may be configured to time optical drive signals and electrode signal sampling to reduce interference.

In particular embodiments, multi-purpose sensors in accordance with the present disclosure provide certain advantages over traditional single-purpose sensors. For example, a combined physiological sensor may employ fewer cables and components than the traditional approach in order to support multiple sensors, which in turn helps reduce cost and total sensor area. A combined physiological sensor may unite multiple sensors within one or more sensor structures and may route all or some sensor output signals to a shared cable connected to one or more monitors. In addition to the simplification, minimization, and cost-reduction offered by a single-cable or reduced number of cables approach, reducing the number of cables for providing information to a monitoring device mitigates the potential for physical interference between cables and monitoring devices. Also provided herein are configurable sensors that include removable portions. In such embodiments, a multi-purpose sensor may include a more long-term portion that may remain in place on the patient and a removable portion that may be detached from the rest of the sensor when its monitoring functionality is no longer as useful for the patient.

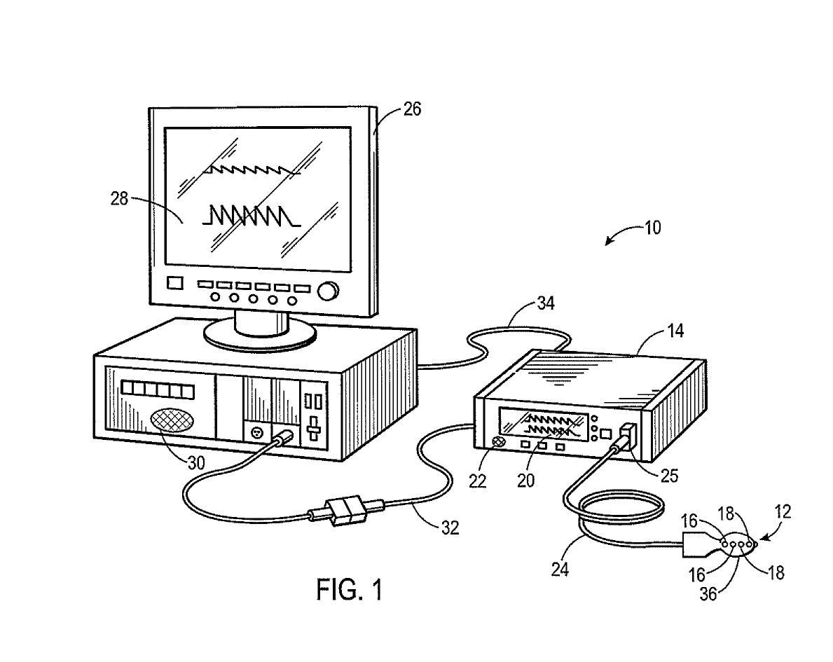

With this in mind, FIG. 1 depicts an embodiment of a patient monitoring system 10 that may be used in conjunction with a medical sensor 12. Although the depicted embodiments relate to sensors for use on a patient's forehead and/or temple, it should be understood that, in certain embodiments, the features of the multi-purpose sensor 12 as provided herein may be incorporated into sensors for use on other tissue locations, such as the finger, the toes, the heel, the ear, or any other appropriate measurement site. In addition, although the embodiment of the patient monitoring system 10 illustrated in FIG. 1 relates to photoplethysmography or pulse oximetry, the system 10 may be configured to obtain a variety of medical measurements with a suitable medical sensor. For example, the system 10 may, additionally be configured to determine patient electroencephalography (e.g., a bispectral index), or any other suitable physiological parameter. As noted, the system 10 includes the sensor 12 that is communicatively coupled to a patient monitor 14. The sensor 12 includes one or more emitters 16 and one or more detectors 18. The emitters 16 and detectors 18 of the sensor 12 are coupled to the monitor 14 via a cable 24 through a plug 25 coupled to a sensor port. Additionally, the monitor 14 includes a monitor display 20 configured to display information regarding the physiological parameters, information about the system, and/or alarm indications. The monitor 14 may include various input components 22, such as knobs, switches, keys and keypads, buttons, etc., to provide for operation and configuration of the monitor. The monitor 14 also includes a processor that may be used to execute code such as code for implementing the techniques discussed herein.

The monitor 14 may be any suitable monitor, such as a pulse oximetry monitor available from Nellcor Puritan Bennett LLC. Furthermore, to upgrade conventional operation provided by the monitor 14 to provide additional functions, the monitor 14 may be coupled to a multi-parameter patient monitor 26 via a cable 32 connected to a sensor input port or via a cable 34 connected to a digital communication port. In addition to the monitor 14, or alternatively, the multi-parameter patient monitor 26 may be configured to calculate physiological parameters and to provide a central display 28 for the visualization of information from the monitor 14 and from other medical monitoring devices or systems. The multi-parameter monitor 26 includes a processor that may be configured to execute code. The a multi-parameter monitor 26 may also include various input components 30, such as knobs, switches, keys and keypads, buttons, etc., to provide for operation and configuration of the a multi-parameter monitor 26. In addition, the monitor 14 and/or the multi-parameter monitor 26 may be connected to a network to enable the sharing of information with servers or other workstations. In one embodiment, the sensor 12 may include a sensor body 36 housing the optical components (e.g., an emitter for emitting light at certain wavelengths into a tissue of a patient and a detector for detecting the light after it is reflected and/or absorbed by the blood and/or tissue of the patient) of the sensor. The sensor body 36 may be formed from any suitable material, including rigid or conformable materials, such as fabric, paper, tubber or elastomeric compositions (including acrylic elastomers, polyimide, silicones, silicone rubber, celluloid, PMDS elastomer, polyurethane, polypropylene, acrylics, nitrile, PVC films, acetates, and latex).

In certain embodiments, the sensor 12 may be a wireless sensor 12. Accordingly, the wireless sensor 12 may establish a wireless communication with the patient monitor 14 and/or the multi-parameter patient monitor 26 using any suitable wireless standard. By way of example, the wireless module may be capable of communicating using one or more of the ZigBee standard, WirelessHART standard, Bluetooth standard, IEEE 802.11x standards, or MiWi standard. In embodiments in which the sensor 12 is configured for wireless communication, the strain relief features of the cable 24 may be housed in the sensor body 36.

As provided herein, the sensor 12 may be a multi-purpose sensor suitable for detection of a plurality of physiological parameters. The sensor 12 may include optical components (e.g., one or more emitters 16 and detectors 18). In one embodiment, the sensor 12 may be configured for photo-electric detection of blood and tissue constituents. For example, the sensor 12 may include pulse oximetry sensing functionality for determining the oxygen saturation of blood as well as other parameters from the plethysmographic waveform detected by the oximetry technique. An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured and other physiological parameters such as the pulse rate and when each individual pulse occurs. Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. At least two, e.g., red and infrared (IR), wavelengths may be used because it has been observed that highly oxygenated blood will absorb relatively less red light and more infrared light than blood with a lower oxygen saturation. However, it should be understood that any appropriate wavelengths, e.g., green, etc., may be used as appropriate. Further, photoplethysmography measurements may be determined based on only one or three or more wavelengths of light.

In addition, the sensor 12 may include regional oximetry functionality. In regional oximetry, by comparing the intensities of at least two wavelengths of light, it is possible to estimate the blood oxygen saturation of hemoglobin in a region of a body. Whereas pulse oximetry measures blood oxygen based on changes in the volume of blood due to pulsing tissue (e.g., arteries), regional oximetry typically examines blood oxygen saturation within the venous, arterial, and capillary systems within a region of a patient. For example, a regional oximeter may include a sensor to be placed on a patient's forehead and may be used to calculate the oxygen saturation of a patient's blood within the venous, arterial and capillary systems of a region underlying the patient's forehead (e.g., in the cerebral cortex). The sensor may include two emitters (e.g., for emitting two wavelengths of light) and two detectors: one detector that is relatively “close” to the two emitters and another detector that is relatively “far” from the two emitters. Light intensity of multiple wavelengths may be received at both the “close” and the “far” detectors. For example, if two wavelength were used, the two wavelengths may be contrasted at each location and the resulting signals may be contrasted to arrive at a regional saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium). Surface data from the skin and skull is subtracted out, to produce an rSO₂ value for deeper tissues. Other methods to calculate regional blood oxygen saturation, such as those provided in U.S. Pat. Nos. 5,139,025 and 5,217,013 or U.S. Patent Publication No. 20110112387, filed Nov. 12, 2009, the disclosures of which are incorporated by reference in their entirety herein for all purposes, may be employed.

In addition to pulse oximetry and regional saturation measurements, the multi-purpose sensors 12 as provided may be configured to monitor other physiological parameters via the optical elements that are configured for pulse oximetry and/or regional saturation monitoring. For example, the sensor 12 may also be configured to detect respiration rate, continous non-invasive blood pressure (CNIBP), tissue water fraction, water fractions, hematocrit, carboxyhemoglobin, met-hemoglobin, total hemoglobin, fractional hemoglobin, intravascular dyes, and/or water content. In addition, the sensor 12 may include additional functionality, such as temperature or pressure sensing functionality.

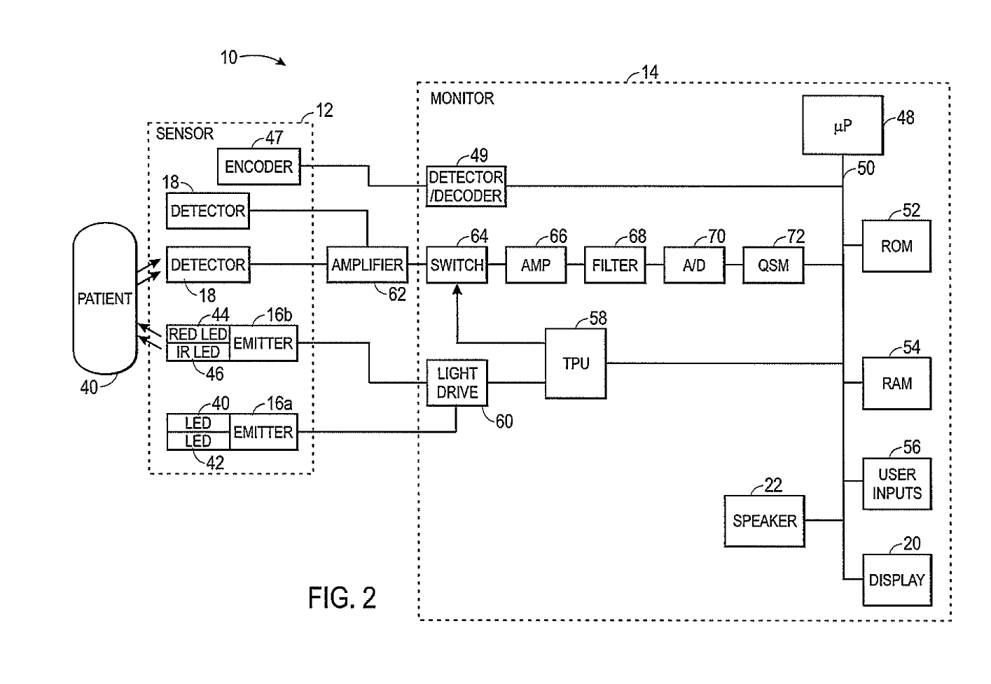

Turning to FIG. 2, a simplified block diagram of the medical system 10 is illustrated in accordance with an embodiment. The sensor 12 may include optical components in the forms of emitters 16 and detectors 18. The emitter 16 and the detector 18 may be arranged in a reflectance or transmission-type configuration with respect to one another. However, in embodiments in which the sensor 12 is configured for use on a patient's forehead, the emitters 16 and detectors 18 may be in a reflectance configuration. An emitter 16 may also be a light emitting diode, superluminescent light emitting diode, a laser diode or a vertical cavity surface emitting laser (VCSEL). An emitter 16 and detector 18 may also include optical fiber sensing elements. An emitter 16 may include a broadband or “white light” source, in which case the detector could include any of a variety of elements for selecting specific wavelengths, such as reflective or refractive elements or interferometers. These kinds of emitters and/or detectors would typically be coupled to the sensor 12 via fiber optics. Alternatively, a sensor assembly 10 may sense light detected from the tissue is at a different wavelength from the light emitted into the tissue. Such sensors may be adapted to sense fluorescence, phosphorescence, Raman scattering, Rayleigh scattering and multi-photon events or photoacoustic effects. In one embodiment, the emitter 16a may be configured for use in a regional saturation technique. To that end, the emitter 16a may include two light emitting diodes (LEDs) 40 and 42 that are capable of emitting at least two wavelengths of light, e.g., red or near infrared light. In one embodiment, the LEDs emit light in the range of 600 nanometers to about 1000 nm. In a particular embodiment, the one LED 40 is capable of emitting light at 730 nm and the other LED is capable of emitting light at 810 nm.

In addition, the emitter 16b may be configured for traditional pulse oximetry. It should be noted that the emitter 16b may be capable of emitting at least two wavelengths of light, e.g., red and infrared (IR) light, into the tissue of a patient, where the red wavelength may be between about 600 nanometers (nm) and about 700 nm, and the IR wavelength may be between about 800 nm and about 1000 nm. The emitter 16b may include a single emitting device, for example, with two LEDs 44 and 46, or the emitter 16b may include a plurality of emitting devices with, for example, multiple LED's at various locations. In some embodiments, the LEDs of the emitter 16b (or 16a) may emit three or more different wavelengths of light. Such wavelengths may include a red wavelength of between approximately 620-700 nm (e.g., 660 nm), a far red wavelength of between approximately 690-770 nm (e.g., 730 nm), and an infrared wavelength of between approximately 860-940 nm (e.g., 900 nm). Other wavelengths may include, for example, wavelengths of between approximately 500-600 nm and/or 1000-1100 nm. Regardless of the number of emitting devices, light from the emitter 16b may be used to measure, for example, oxygen saturation, water fractions, hematocrit, or other physiologic parameters of the patient. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In any suitable configuration of the sensor 12, the detectors 18a and 18b may be an array of detector elements that may be capable of detecting light at various intensities and wavelengths. In one embodiment, light enters the detector 18 (e.g., detector 18a or 18b) after passing through the tissue of the patient. In another embodiment, light emitted from the emitter 16 may be reflected by elements in the patent's tissue to enter the detector 18. The detector 18 may convert the received light at a given intensity, which may be directly related to the absorbance and/or reflectance of light in the tissue of the patient, into an electrical signal. That is, when more light at a certain wavelength is absorbed, less light of that wavelength is typically received from the tissue by the detector 18, and when more light at a certain wavelength is reflected, more light of that wavelength is typically received from the tissue by the detector 18. After converting the received light to an electrical signal, the detector 18 may send the signal to the monitor 14, where physiological characteristics may be calculated based at least in part on the absorption and/or reflection of light by the tissue of the patient.

In certain embodiments, the medical sensor 12 may also include an encoder 47 that may provide signals indicative of the wavelength of one or more light sources of the emitter 16, which may allow for selection of appropriate calibration coefficients for calculating a physical parameter such as blood oxygen saturation. The encoder 47 may, for instance, be a coded resistor, EEPROM or other coding devices (such as a capacitor, inductor, PROM, RFID, parallel resident currents, or a colorimetric indicator) that may provide a signal to a microprocessor 48 related to the characteristics of the medical sensor 12 to enable the microprocessor 48 to determine the appropriate calibration characteristics of the medical sensor 12. Further, the encoder 47 may include encryption coding that prevents a disposable part of the medical sensor 12 from being recognized by a microprocessor 48 unable to decode the encryption. For example, a detector/decoder 49 may translate information from the encoder 50 before it can be properly handled by the processor 48. In some embodiments, the encoder 47 and/or the detector/decoder 48 may not be present.

Signals from the detector 18 and/or the encoder 47 may be transmitted to the monitor 14. The monitor 14 may include one or more processors 48 coupled to an internal bus 50. Also connected to the bus may be a ROM memory 50, a RAM memory 54 and a display 20. A time processing unit (TPU) 58 may provide timing control signals to light drive circuitry 60, which controls when the emitter 16 is activated, and if multiple light sources are used, the multiplexed timing for the different light sources. It is envisioned that the emitters 16a and 16b may be controlled via time division multiplexing of the light sources. TPU 58 may also control the gating-in of signals from detector 18 through a switching circuit 64. These signals are sampled at the proper time, depending at least in part upon which of multiple light sources is activated, if multiple light sources are used. The received signal from the detector 18 may be passed through an amplifier 66, a low pass filter 68, and an analog-to-digital converter 70 for amplifying, filtering, and digitizing the electrical signals the from the ear sensor 12. The digital data may then be stored in a queued serial module (QSM) 72, for later downloading to RAM 52 as QSM 72 fills up. In an embodiment, there may be multiple parallel paths for separate amplifiers, filters, and A/D converters for multiple light wavelengths or spectra received.

In an embodiment, based at least in part upon the received signals corresponding to the light received by detector 18, processor 48 may calculate the oxygen saturation using various algorithms. These algorithms may use coefficients, which may be empirically determined. For example, algorithms relating to the distance between an emitter 16 and various detector elements in a detector 18 may be stored in a ROM 52 and accessed and operated according to processor 48 instructions.

Furthermore, one or more functions of the monitor 14 may also be implemented directly in the sensor 12. For example, in some embodiments, the sensor 12 may include one or more processing components capable of calculating the physiological characteristics from the signals obtained from the patient. In accordance with the present techniques, the sensor 12 may be configured to provide optimal contact between a patient and the detector 18, and/or the emitter 16. The sensor 12 may have varying levels of processing power, and may output data in various stages to the monitor 14, either wirelessly or via the cable 24. For example, in some embodiments, the data output to the monitor 14 may be analog signals, such as detected light signals (e.g., pulse oximetry signals or regional saturation signals), or processed data.

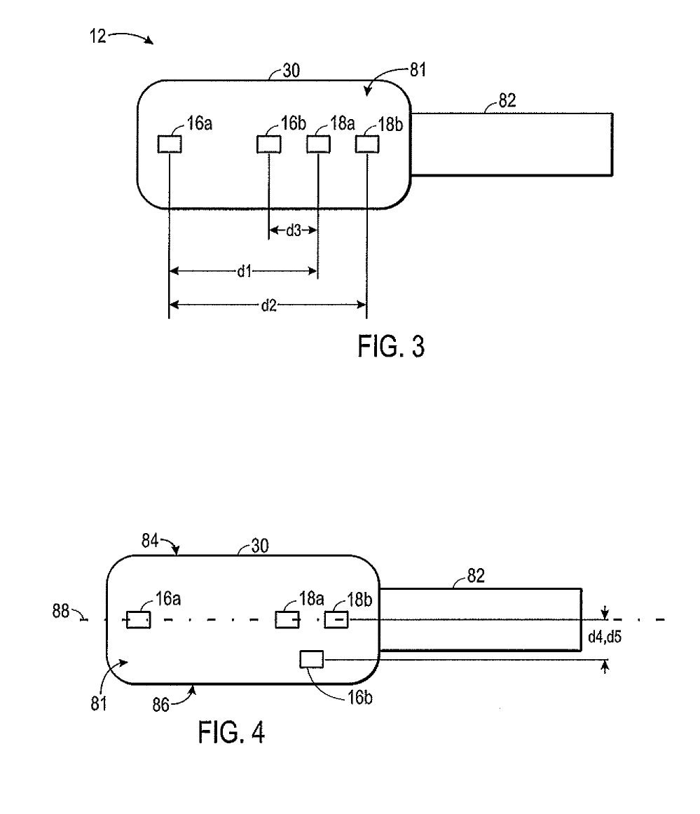

As discussed herein, pulse blood oxygen saturation and regional blood oxygen saturation may be measured and/or calculated with a multi-purpose sensor 12. FIG. 3 is an example of a sensor 12 that includes optical elements arranged in a configuration on a sensor body 30 on a tissue-contacting surface 81 to facilitate both types of monitoring in a single sensor. The sensor body 82 may be constructed from suitable materials that may conform to the patient's forehead. The emitter 16a is configured to be used with the detectors 18a and 18b for regional saturation measurements while the emitter 16b is configured to be used with the detector 18 for pulse oximetry measurements. In the depicted configuration, the emitter 16a is a first distance, d₁, from the detector 18a and a second distance, d₂, from the detector 18b. In addition, the sensor 12 includes an emitter 16b configured for pulse oximetry. The emitter 16b is positioned a distance d₃ from the detector 18a. The distance d₃ is shorter than both d₁ and d₂.

For regional saturation measurements, the emitter-detector spacing d₁ represents a shallower optical path and the emitter-detector spacing d₂ represents a deeper optical path for cranial penetration. Accordingly, distance d1 is shorter than distance d₂. In certain embodiments, distance d₁ is about 75% of the distance d₂. In a particular embodiment, distance d₁ is about 30 mm and distance d₂ is about 40 mm. In other embodiments, d₁ may be 1-3 centimeters and d₂ may be 3-4 centimeters. In a particular embodiment, the emitter 16a and detector 18a and 18b may be configured relative to one another as in the Somanetics INVOS® Cerebral/Somatic Oximeter, which is designed specifically to measure oxygen in brain or tissues directly beneath the sensor using two wavelengths, 730 and 810 nm, to measure changes in regional oxygen saturation (rSO₂ index). For pulse oximetry measurements, the optical path is designed to penetrate the layer of skin on the forehead to capture oxygen saturation of the vessels of the forehead. Accordingly, the emitter-detector spacing d₃ is less than d₁ or d₂. In one embodiment, the distance d₃ is less than half the distance d₁. In particular embodiments, the distance d₃ is about 10 mm.

Further, detector 18a may be smaller in size than light detector 18b in order to equalize the differences in light intensity received/detected due to the distance of the detector from emitters 16a. The size of a detector may be a function of the distance of the detector from an emitter. The sensor 12 may also include features for blocking unwanted light infiltration, such as light block 82. The size and/or the distance of a detector from an emitter may be a function of a desired mean path length of light traversing through human tissue. Although the use of two detectors and two emitters are depicted, any suitable number of detectors 18 or emitters 16 may be used that provide the appropriate emitter-detector spacing. For example, four detectors may be used, each positioned at a different distance d from emitter 16a. Sensor 12 may be configured to use a particular subset of the multiple detectors 18 depending on the clinical condition of the patient. It should also be understood that the positions of the emitters 16 and detectors 18 may be exchanged so long as appropriate emitter-detector spacing is maintained.

Arranging the emitters 16 and detectors 18 along an axis may provide certain manufacturing advantages and may reduce the complexity of routing electrical leads through a shared cable (e.g., cable 24). However, in certain embodiments, it may be advantageous to position the pulse oximetry-configured emitter 16b such that, when the sensor 12 is applied, the emitter 16b is relatively closer to the patient's lower forehead region. For example, the tissue in the lower forehead region may have relatively better blood perfusion characteristics that may lead to improved oximetry measurements. In addition, the optical elements for regional saturation monitoring may be configured to be positioned higher on the forehead for improved cranial penetration. By positioning optical elements on the sensor such, when the sensor is applied, an oximetry optical element is relatively lower and the regional saturation optical elements are relatively higher on the forehead, the advantages of combination are achieved while maintaining appropriate positioning of the emitters 16 and detectors 18. As shown in FIG. 4, the sensor 12 may include a top edge 84 that is configured for placement closer to the scalp or top of the head and a lower edge 86 that is positioned closer to the eyebrow. The pulse oximetry emitter 16b may be disposed on the tissue-contact surface 81 below the detectors 18. In embodiments in which the regional saturation optical elements are arranged generally along an axis 88, the pulse oximetry emitter 16b may be located off the axis 88. The exterior surface of the sensor 12 may include additional positioning features, such as alignment or positioning indicators, to facilitate correct application of the top edge 84 and the bottom edge 86 on the patient.

In one embodiment, the emitter 16b may be located relative to the detectors 18a and 18b such that the distance d₄ from emitter 16b—detector 18a and the distance d₅ from emitter 16b—detector 18b are substantially equal. In another embodiment, the distance d₄ and the distance d₅ may be different. For example, in one embodiment, d₄ may be about 8 mm and d5 may be about 12 min. In this manner, the monitor 14 may be configured to receive signals from both detectors 18 and evaluate which signal should be used to determine physiological parameters based on the more optimal emitter-detector spacing or best signal-to-noise ratio, which may vary from patient to patient and may depend on the patient's anatomical features, skin pigmentation, clinical condition, sensor adhesion, etc. Further, the monitor 14 may be configured to receive both signals and combine the signals (e.g., perform averaging) or arbitrate based on signal quality to calculate the oxygen saturation and/or heart rate.

While certain disclosed embodiments include multi-purpose sensors 12 with optical elements configured for pulse oximetry and/or regional saturation measurements, it is also contemplated that multi-purpose sensors 12 may include BIS monitoring functionality. In particular, BIS sensors are often applied to a patient's forehead during surgical procedures. Because BIS sensors have multiple electrodes that are designed to be placed in particular locations on the forehead, the presence of other sensors on the forehead may interfere with proper positioning of the BIS sensor. By combining the types of medical sensors that may be applied to a patient's forehead into a single sensor, the multi-purpose sensors 12 provided herein may facilitate proper positioning of BIS electrodes. Further, frontal EEG electrodes, such as those used in BIS monitoring, may be more sensitive to certain types of events than regional saturation monitoring. Accordingly, combining regional saturation monitoring and BIS monitoring may provide improved measurements of particular physiological indicators.

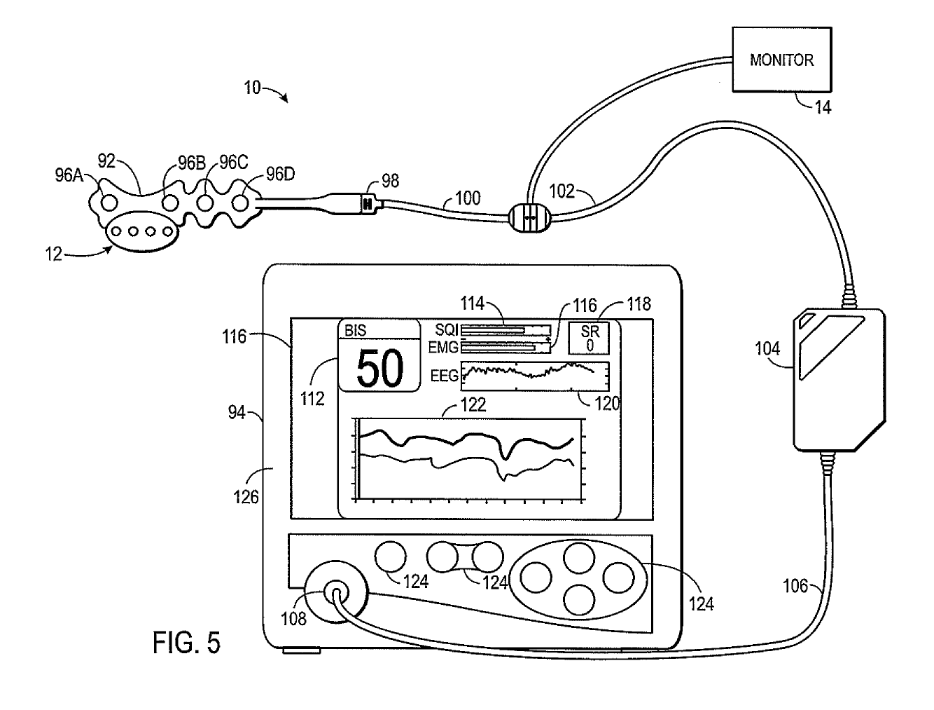

With the foregoing in mind, FIG. 5 is a front view of an embodiment of a 10 coupled to the sensor 12 and an EEG monitor 94. The EEG monitor 94 may be incorporated into the patient monitor 14 or may be a standalone device. The sensor 12 may include electrodes 96 (e.g., four electrodes 96a, 96b, 96c, and 96d) that are self adherent and self prepping to temple and forehead areas of a patient and that are used to acquire EEG signals. The sensor 12 may be coupled through connector 98 to a cable 100 (e.g., patient interface cable), which in turn may be coupled to a cable 102 (e.g., pigtail cable). In certain embodiments, the sensor 12 may be coupled to the cable 102 thereby eliminating the cable 100. Further, in embodiments in which the EEG monitor 94 is a standalone device, the cable 102 may split and may include a portion that coupled the optical elements of the sensor 12 to the patient monitor 14. The cable 102 may be coupled to a digital signal converter 104, which in turn is coupled to the cable 106 (e.g., monitor interface cable). In certain embodiments, the digital signal converter 104 may be embedded in the EEG monitor 94 to eliminate the cables 102 and 26. Cable 106 may be coupled to the EEG monitor 94 via a port 108 (e.g., digital signal converter port).

The EEG monitor 94 may be capable of calculating physiological characteristics relating to the EEG signal received from the sensor 12. For example, the monitor may be capable of algorithmically calculating BIS from the EEG signal. BIS is a measure of a patient's level of consciousness during general anesthesia. Techniques for BIS monitoring may be as provided in U.S. Provisional Application No. 61/301,088, filed Feb. 3, 2010, and U.S. patent application Ser. No. ______, “Combined Physiological Sensor Systems and Methods,” which are hereby incorporated by reference herein in their entirety for all purposes. Further, the EEG monitor 94 may include a display 110 capable of displaying the physiological characteristics, historical trends of physiological characteristics, other information about the system (e.g., instructions for placement of the sensor 12 on the patient), and/or alarm indications. The EEG monitor 94 may display a patient's BIS value 112. The BIS value 112 represents a dimensionless number (e.g., ranging from 0, i.e., silence, to 100, i.e., fully awake and alert) output from a multivariate discriminate analysis that quantifies the overall bispectral properties (e.g., frequency, power, and phase) of the EEG signal. For example, a BIS value 112 between 40 and 60 may indicate an appropriate level for general anesthesia. The EEG monitor 94 may also display a signal quality index (SQI) bar graph 114 (e.g., ranging from 0 to 100) which measures the signal quality of the EEG channel source(s) based on impedance data, artifacts, and other variables. The EEG monitor 94 may yet also display an electromyograph (EMG) bar graph 116 (e.g., ranging from 30 to 55 decibels) which indicates the power (e.g., in decibels) in the frequency range of 70 to 110 Hz. The frequency range may include power from muscle activity and other high-frequency artifacts. The EEG monitor 94 may further display a suppression ratio (SR) 118 (e.g., ranging from 0 to 100 percent) which represents the percentage of epochs over a given time period (e.g., the past 63 seconds) in which the EEG signal is considered suppressed (i.e., low activity). In certain embodiments, the EEG monitor 94 may also display a burst count for the number of EEG bursts per minute, where a “burst” is defined as a short period of EEG activity preceded and followed by periods of inactivity or suppression. The EEG monitor 94 may yet further display the EEG waveform 120. In certain embodiments, the EEG waveform 120 may be filtered. The EEG monitor 94 may still further display trends 122 over a certain time period (e.g., one hour) for EEG, SR, EMG, SQI, and/or other parameters. As described below, in certain embodiments, the EEG monitor 94 may display stepwise instructions for placing the sensor 12 on the patient. In addition, the EEG monitor 94 may display a verification screen verifying the proper placement of each electrode 96 of the sensor 12 on the patient. In certain embodiments, the monitor 12 may store instructions on a memory specific to a specific sensor type or model. In other embodiments, the sensor 12 may include a memory that provides the instructions to the EEG monitor 94.

Additionally, the EEG monitor 94 may include various activation mechanisms 124 (e.g., buttons and switches) to facilitate management and operation of the EEG monitor 94. For example, the EEG monitor 94 may include function keys (e.g., keys with varying functions), a power switch, adjustment buttons, an alarm silence button, and so forth. It should be noted that in other embodiments, the parameters described above and the activation mechanisms 124 may be arranged on different parts of the EEG monitor 94. In other words, the parameters and activation mechanisms 124 need not be located on a front panel 126 of the EEG monitor 94. Indeed, in some embodiments, activation mechanisms 124 are virtual representations in a display or actual components disposed on separate devices. In addition, the activation mechanisms 124 may allow selecting or inputting of a specific sensor type or model in order to access instructions stored within the memory of the EEG monitor 94.

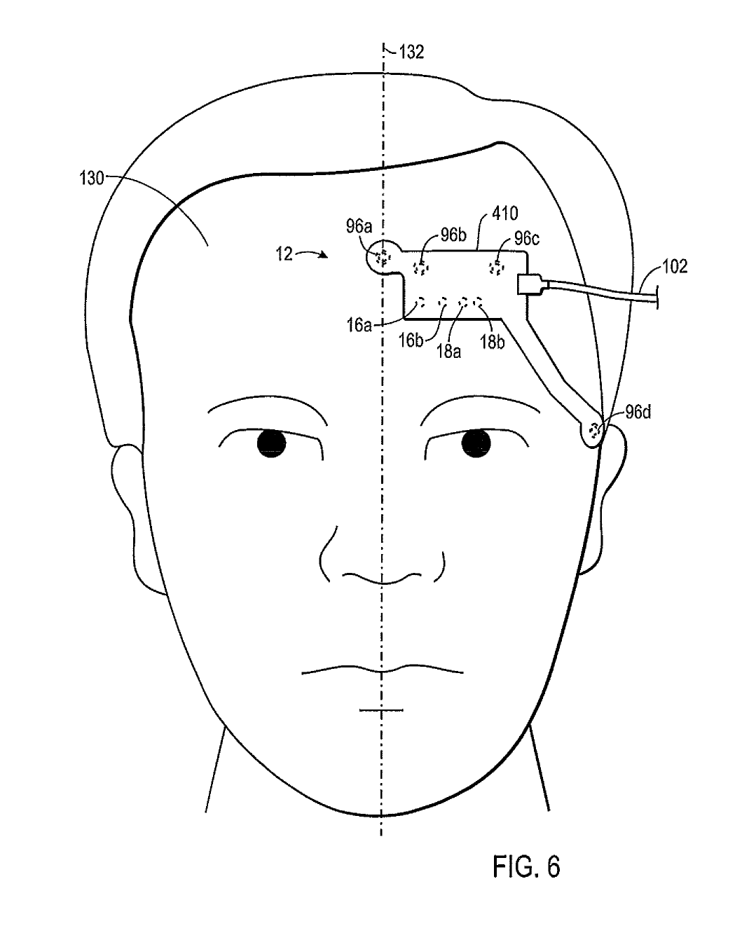

FIG. 6 shows an illustrative multi-purpose sensor 12 applied to a patient, in accordance with an embodiment. While the depicted arrangement shows four EEG electrodes 96, fewer or more electrodes 96 may be included. In the arrangement depicted in FIG. 6, the electrodes 96a and 96b are generally close to the center axis 132 of the forehead 130 of the patient, while electrode 96 is positioned to the upper side of the forehead and electrode 96d is positioned over the temple that is closest to the other electrodes 96. In particular arrangements, electrode 96d may have a dedicated cable. In the depicted arrangement, the electrode 96d is shown incorporated into the same sensor structure (i.e., sensor body 30) as the other electrodes. In other arrangements, however, the electrode 96 may part of a second but connected sensor structure (e.g., flex circuit) that connects to sensor body 30 via cable or other flexible connection means, electrode 96d may communicate with a wireless receiver (not shown) via a wireless transmitter (not shown).

The design or shape of the embodiments of the sensors 12 that include BIS monitoring functionality may include features to help facilitate the proper placement of the electrodes 96, and thus the sensor 12 and any associated emitters 16 and detectors 18 on the patient's temple and forehead. For example, the electrodes 96 may be at fixed distances with respect to each other to allow for conformity in the diagonal placement of the electrodes 96. In particular, the electrodes 96A and 96B may include a bridge of sufficient width and rigidity to fix the distance between these electrodes 96A and 96B and to prevent lateral movement along a longitudinal axis of the sensor 12. Also, the bridge may include a curvature of a concave nature configured to trace up and around the lateral and top edges of the patient's eyebrow to reinforce correct placement of the electrodes 96A and 96B. In addition, the sensor 12 may include labels or other features (e.g., arrows) to facilitate the proper placement of the electrodes 96. As shown in FIG. 5, the emitters 16 and the detectors 18 are positioned below the electrodes along an axis that is above the eyebrow of subject. The emitters 16 and the detectors 18 may be positioned in alternative locations, although sites of highly perfused tissue (such as the forehead, above the eyebrow) are suitable for measuring blood oxygen saturation.

In addition, the design or shape of the sensor 12 may include features to prevent the sensor from lifting from the patient's skin. For example, the areas of sensor 12 surrounding one or more electrodes 16 may include protrusions or tabs to counteract peeling forces and to reduce adhesion shear. Also, a tail section of the sensor 12 configured to connect with the cables may include a narrow tail section to prevent the twisting of the tail section and the potential marking of the patient's skin. The electrodes 96 may be formed from a suitable conductive composition, such as a metal or alloy (e.g., silver/silver chloride, copper, aluminum, gold, or brass) or a conductive polymer (such as screen-printable silver/silver chloride inks carbon impregnated polymers).

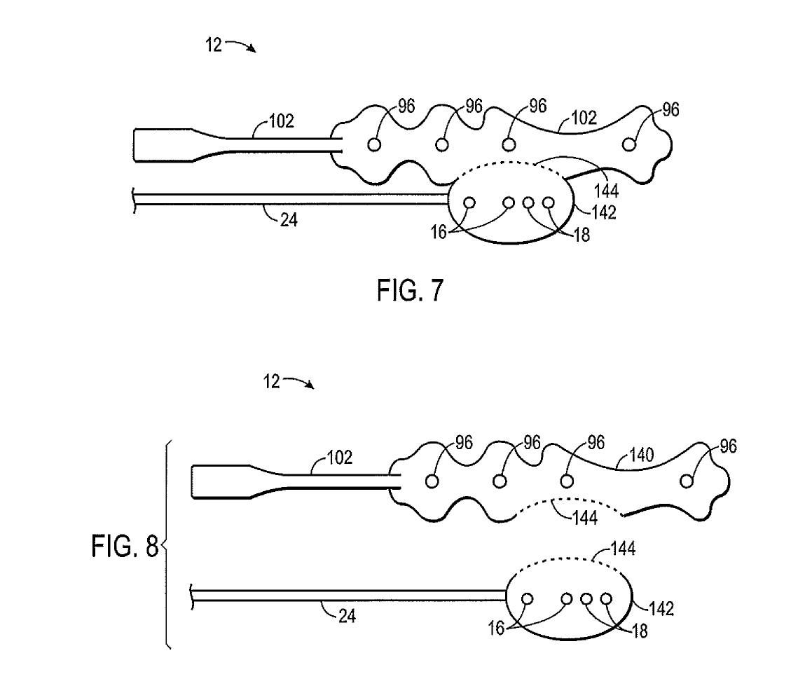

While combination sensors 12 provide advantages related to ease of application and a consolidation of cables that extend from the patient bedside, in certain embodiments, portions of the sensor 12 may become less relevant to the patient's clinical condition. To that end, provided herein are multi-purpose sensors that include one or more removable portions. Accordingly, after use, a clinician may remove a portion of the sensor 12 that is no longer needed and leave in place another portion of the sensor 12 for continued monitoring of the desired physiological parameters. Illustrated in FIG. 7 is the sensor 12 in which the portion 140 is capable of being removed from the portion 142. Here, the electrodes 96 are contained within the portion 140 and the optical elements are contained within the portion 142. To facilitate removal of the portion 140, each portion (e.g., portions 140 and 142) may be formed with a dedicated cable (e.g., cables 24 and 102) so that the remaining portion of the sensor 12 on the patient has continued electrical coupling to the system. Alternatively, the sensor 12 may be formed with a single cable that extends from the portion designed to be left in place on the patient. For example, in one embodiment, a single cable extends from the portion 142 and is coupled to the electrodes 96 such that removal of the BIS monitoring portion 142 breaks its connection to the cable without interfering with the connection of the optical elements to the cable.

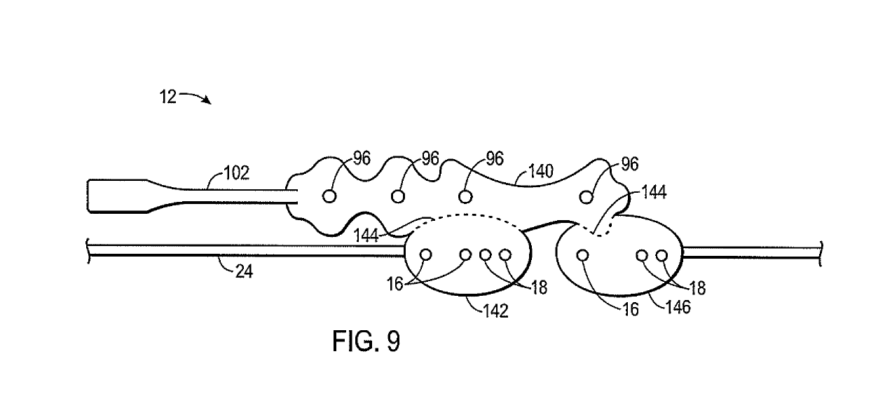

Because, in particular embodiments, BIS monitoring may take place during surgery or while the patient is anesthetized, the clinician may wish to remove the BIS monitoring portion 140 after the patient comes out of anesthesia. Removal of the BIS monitoring portion may also allow the caregiver to clean off any electrode gel, which may irritate the patient's skin over time. In addition, removal of portions of the sensor 12 may unambiguously mark the sensor 12 as used, which may prevent inappropriate reuse of disposable sensors. It should be understood that the sensor 12 may be configured in any appropriate configuration depending on the portion that is removed. FIG. 8 shows an embodiment in which portion 140 has been removed from portion 142, for example by tearing along perforation 144. In other embodiments, the removable portion 140 may be cut away from the portion 142 or parts of the sensor 12 including a backing and the electrodes 96 may be peeled away. The portion 142 retains the ability to measure oxygen saturation and regional saturation because the emitters 16 and the detectors 18 are all included in the portion 142. FIG. 9 illustrates an embodiment in which the sensor 12 includes three portions, 140, 142, and 146, any of which can be removed from another. Portion 146 may be configured for regional saturation measurements while portion 142 is configured for combination pulse oximetry and regional saturation measurements. It should also be understood that portion 142 may include subportions, such that all or part of the regional saturation portion (e.g., an emitter coupled to two detectors) may be removed from the pulse oximetry portion (e.g., a second emitter).

The monitor 14 may be configured to determine whether the sensor 12 is in the intact configuration. In one embodiment, a user may input information about the configuration of the sensor 12 into a medical device, such as the monitor 14. For example, a user may input that the sensor 12 no longer includes the portion 140 for BIS monitoring or the portion 142 is for combination pulse oximetry and regional saturation measurements. In other embodiments, the monitor 14 may be configured to determine if any portion of the sensor has been removed based on the presence or absence of expected signals from the detectors 18 or the electrodes 96. In one embodiment, the monitor 14 may continue to provide monitoring for the remaining portion of the sensor after removal, even if other portions have been removed and the monitor 14 has determined that the sensor 12 is no longer intact, i.e., the BIS sensor is in the sensor off configuration. In specific embodiments, the monitor 14 may provide an alarm or other indicator to alert the caregiver that a portion of the sensor is either off or has been deliberately removed. This alert may prevent confusion between a deliberate removal of part of the sensor and an accidental dislodgement of part of the sensor. In other embodiments, the sensor 12 may provide an indicator, such as an electrical signal (or the absence of one), to the monitor 14 that may indicate if a portion of the sensor (e.g., portion 140 or 142) has been removed. The sensor body 30 may include one or more elements configured to provide such feedback to the monitor 14, such as electrical elements, transmitters or receivers (e.g., RFID tags associated with a removable portion or attached portion of the sensor 12), related to the presence or absence of any portion of the sensor 12. For example, removal of a portion may break an indicator circuit that passes through the removal indicator (e.g., perforation line 144) and that is closed when the portion 140 is still attached to the portion 142. When one portion is removed, the circuit may be broken. Generally, it is envisioned that the indicator circuit may include a conductive material that is either easily broken by hand or is easily cut by scissors. For example, the indicator circuit may include thin foil material or a thin semiconductive ceramic in the portion of the circuit that connects the portion 140 and the portion 142.

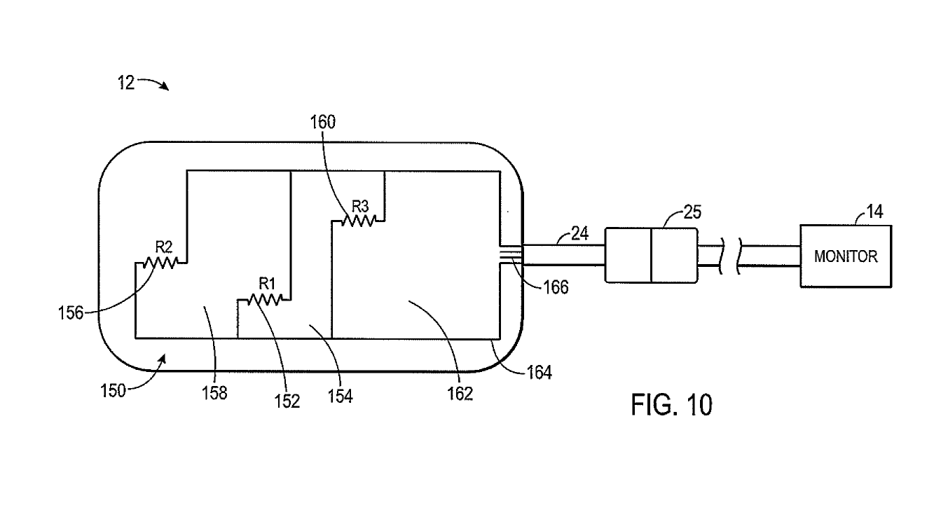

In other embodiments, the electrical feedback from the sensor 12 may be based on an initial resistance versus a measured resistance. For example, as shown in FIG. 10, the sensor 12 may include a set of N parallel resistors (e.g., resistors 152, 156, and 160) in a circuit 150 such that, when an intact sensor 12 is connected to the monitor 14, the monitor is able to determine that the initial resistance of the circuit is R=1/(1/R1+1/R2++1/R3+ . . . +1/Rn). In the depicted embodiment, N=3, and the initial resistance is calculated as R=1/(1/R1+1/R2+1/R3). However, it should be understood that other resistor arrangements may be implemented. Removal of portions of the sensor 12 that include a part of the circuit 150 change the total resistance of the circuit 150. For example, removal of portion 158 from portions 154 and 162 changes the total resistance. If the values for the resistors are suitably chosen (e.g. different for each removable portion of the sensor 12 and such that each combination of resistors yields a unique value), the monitor 14 may determine how many and what type of sensor portions are connected by measuring a single resistance, e.g. via only one wire and connector pin in connector 25, plus an existing ground 164 through cable 24 that also includes other wires leads from the optical or electrode elements of the sensor 12. Suitable techniques for determining identity of a sensor via resistance values may be as provided in U.S. Pat. No. 4,770,179, the disclosure of which is incorporated by reference in its entirety herein for all purposes. When one portion of the sensor 12 is torn away, the resistor goes with the discarded sensor and the monitor 14 may be configured to periodically read the total resistance to detect which portion of the sensor 12 has been removed and which portions of the sensor 12 are remaining. Alternatively, one wire memory chips may be detected on a one-wire serial bus, and the configuration of the sensor 12 may be measured this way.

As provided herein, the multi-purpose sensors 12 may include features for reducing cross-talk and/or interference between the optical components, such as the emitter 16 and detector 18, and the electrodes 96. The LED drive signals of the emitters 16 may produce artifacts in the EEG signals, which are in the microvolt range. In one embodiment, cross-talk may be reduced by providing shielding for the electrodes 96. For example, the sensor 12 may include shielding layer—between tissue contact surface 81 and the exterior surface—to reduce interference between the two sets of signals. For example, the intervening layer may be a non-conductive or dielectric material, or the intervening layer may be connected to electrical ground (e.g., via a ground wire provided within cable 24). Furthermore, the length of interconnects may be minimized to reduce the amount of conductive material associated with the electrodes. Additionally, modifications to the positioning of the optical components and the electrodes 96 on the sensor body 30 may provide additional noise reduction. In other embodiments, the monitor 14 (or 94) may be configured to reduce the effects of any interference. For example, the monitor 14 may be capable of time division multiplexing, and the signals acquired by the electrodes 96 may be processed only during times when the emitters 16 are not activated. In other embodiments, the monitor 14 may be capable of frequency division multiplexing.

In addition, the present disclosure includes suitable techniques for fabricating sensors 12 as provided herein. In one embodiment, the combination sensors 12 are manufactured as a unitary assembly. For example, the sensor body 30 may be formed from a suitable based material, such as a printed circuit sheet or board, onto which the electrodes 96 are formed at appropriate spacing. The printed circuit sheet may also include connectors located at appropriate spacing for electrical connection of emitters 16 and detector 18. Any intervening layers, such as layers that protect and shield the printed circuit sheet, may then be applied to the assembly. Such layers may include apertures configured to accommodate the emitters 16 and detectors 18, which may be placed into the apertures. Further, the electrical connectors designed to couple the electrodes 96 and optical elements may be positioned on the sensor body 30 such that removal of particular portions will leave the coupling to the remaining portions intact.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims

What is claimed is:

1. A combination sensor, comprising:
a sensor body having a tissue contact surface.
first optical elements configured for regional saturation monitoring disposed on the tissue contact surface of the sensor body;
second optical elements configured for pulse oximetry monitoring disposed on the tissue contact surface of the sensor body; and
a plurality of electrodes configured for bispectral index monitoring disposed on the tissue contact surface of the sensor body.

2. The sensor of claim 1, wherein the sensor body is configured to be applied to a patient's forehead.

3. The sensor of claim 1, wherein a first portion of the sensor body comprising the first optical elements is capable of being removed from a second portion comprising the second optical elements.

4. The sensor of claim 1, wherein the first optical elements comprise a first emitter and a first photodetector spaced apart from one another a first distance on the tissue contact surface of the sensor body; and
a second photodetector spaced a second distance apart from the first emitter on the tissue contact surface of the sensor body, wherein the second distance is greater than the first distance.

5. The sensor of claim 4, wherein the second optical elements comprise a second emitter having two light emitting diodes, wherein the second emitter is coupled to the first photodetector or the second photodetector and wherein the second emitter is spaced a third distance apart from the first photodetector, wherein the third distance is less than the first distance.

6. The sensor of claim 5, wherein the first distance is at least twice the third distance.

7. The sensor of claim 5, wherein the second emitter is about equidistant from the first detector and the second detector.

8. The sensor of claim 5, wherein the first emitter, the first photodetector, and the second photodetector are disposed along an axis.

9. The sensor of claim 4, wherein the first distance is about 30 mm or greater.

10. The sensor of claim 1, wherein the second optical elements comprise an emitter spaced a distance apart from a photodetector.

11. A monitor configured to be coupled to a combination sensor for pulse oximetry, regional saturation and bispectral index monitoring, the monitor configured to:
drive one or more emitters on the combination sensor configured for pulse oximetry or regional saturation;
receive one or more photoplethysmography signals from one or more photodetectors on the combination sensor configured for pulse oximetry or regional saturation;
receive one or more electroencephalography signals from a plurality of electrodes on the combination sensor configured for bispectral index monitoring; and
determine if any of the one or more emitters, one or more photodetectors, or plurality of electrodes has been removed or disconnected from the sensor such that monitoring functions for one or more of pulse oximetry, regional saturation or bispectral index monitoring have been disabled.

12. The monitor of claim 11, wherein the monitor is configured to determine a bispectral index based on the one or more electroencephalography signals when the plurality of electrodes are coupled to the sensor.

13. The monitor of claim 12, wherein determining the bispectral index comprises eliminating a portion of the one or more electroencephalography signals from a time period when the emitter was activated.

14. The monitor of claim 11, wherein the monitor is configured to determine a regional saturation based on the one or more photoplethysmography signals when at least two photodetectors and at least one emitter are coupled to the sensor.

15. The monitor of claim 11, wherein the monitor is configured to determine a blood oxygen saturation based on the one or more photoplethysmography signals when at least one photodetector and at least one emitter are coupled to the sensor.

16. The monitor of claim 11, wherein the monitor is configured to determine a whether a portion of the sensor has been removed based on the presence of the one or more photoplethysmography signals.

17. The monitor of claim 11, wherein the monitor is configured to determine a whether a portion of the sensor has been removed based on the presence of the one or more photoplethysmography signals.

18. The monitor of claim 11, wherein the monitor is configured to determine a whether a portion of the sensor has been removed based on a user input.

19. The monitor of claim 11, wherein the monitor is configured to determine a whether a portion of the sensor has been removed based on an electrical signal from an element associated with the portion.

20. A unitary sensor assembly, comprising:
a conformable sensor body having a tissue contact surface;
a first electrode and a second electrode disposed on the tissue contact surface of the conformable sensor body;
an emitter and a first photodetector spaced apart from one another a first distance on the tissue contact surface of the conformable sensor body;
a second photodetector spaced a second distance apart from the first emitter, wherein the second distance is greater than the first distance; and
a cable extending from the sensor body and coupled to the first electrode, the second electrode, the emitter, the first photodetector, and the second photodetector.
